(19) United States
(12) Patent Application Publication
(10) Pub. No.: US 2022/0280671 A1
(43) Pub. Date: Sep. 8, 2022

(54) FACILITY IMMUNE SYSTEM

(71) Applicant: Chenghung PAN, Palo Alto, CA (US)

(72) Inventor: Chenghung PAN, Palo Alto, CA (US)

(21) Appl. No.: 17/680,988

(22) Filed: Feb. 25, 2022

Related U.S. Application Data
(60) Provisional application No. 63/155,729, filed on Mar. 2, 2021.
(60) Provisional application No. 63/160,005, filed on Mar. 12, 2021.
(60) Provisional application No. 63/173,655, filed on Apr. 12, 2021.
(60) Provisional application No. 63/184,357, filed on May 5, 2021.

(51) Int. Cl. A61L 2/28; A61L 2/24; A61L 2/10

(52) U.S. Cl. CPC A61L 2/28; A61L 2/24; A61L 2/10; A61L 2202/14; A61L 2202/16; A61L 2202/25

(57) ABSTRACT

A facility immune system (FIS) uses time, occupancy, or sensor data to identify or document areas of a facility that need disinfection and that do not need disinfection. The FIS may employ cells respectively in the areas, and the cells may include status indicators for the areas, beacons that interact with mobile devices, and/or sensor systems to sense human activity or other conditions in the areas. The FIS may further control disinfection units to start and end disinfection processes. The FIS may also communicate with facility staff regarding disinfection processes and may provide an administrative interface that enables an administrator to: monitor infection statuses of areas; control, schedule, or provide instructions for disinfection or cleaning processes; and document disinfection information.

### CROSS-REFERENCE TO RELATED APPLICATIONS

This patent document claims benefit of the earlier filing date of U.S. provisional Pat. App. No. 63/155,729, entitled “An Immune System for Facilities,” filed Mar. 2, 2021, U.S. provisional Pat. App. No. 63/160,005, entitled “A Facility Immune Cell Device,” filed Mar. 12, 2021, U.S. provisional Pat. App. No. 63/173,655, entitled “An Internet-of-Things System with No Security Risk,” filed Apr. 12, 2021, and U.S. provisional Pat. App. No. 63/184,357, entitled “A Facility Immune System,” filed May 5, 2021, which are hereby incorporated by reference in their entirety.

### BACKGROUND

Contamination of facilities with pathogens such as bacteria or viruses can spread infections to humans. To reduce the risk of infections, facilities may need to be cleaned and disinfected to remove contagions, but determining when a facility or an area, an infrastructure, or furnishings at a facility needs to be disinfected is subject to errors. An error that delays a disinfection process may allow contamination to persist and cause infections. To avoid this, disinfection decisions may be biased toward safety and err on the side of disinfecting even when disinfecting is not required. In some cases, disinfection is completely unnecessary and a waste of resources, but unnecessary disinfections are still performed when no proof of safety is available.

An area, infrastructure, or furnishing at a facility, once identified for disinfection, generally needs to be disinfected manually or with significant human planning, supervision, and control. The disinfection techniques also may vary depending on available disinfection equipment and personnel and on the type of area, infrastructure, or furnishing being disinfected. A plan for a disinfection process is generally needed to provide efficient disinfection. Records are also needed to document when and where disinfection processes were completed. Disinfection tasks thus require choices, planning, supervision, and record keeping. For example, an administrator may need to identify areas to be disinfected, choose which resources to employ in which areas, plan how the resources are used, and record when, where, and how disinfection was completed. Monitoring and decontaminating a large facility can therefore be a complex task requiring human labor and supervision, and systems and methods that improve or simplify the monitoring and decontaminating a facility are desired.

The disinfection of aerosols may be even more critical than the disinfection of surfaces in the case of an aerosol transmitted disease, such as TB, Influenza, COVID-19, and its variants. Heating Ventilation and Air Conditioning (HVAC) and air purifiers systems may include filters or disinfectors that remove aerosols. However, when people gather in an area, infectious aerosol concentration may build up faster than available HVAC systems and air purification systems can remove the infectious aerosols, so that the infectious aerosol concentration in the area may build-up and exceed an infection threshold concentration at which infections become likely. Facility systems and processes are also needed to control aerosols and keep the worst-case concentration of the infectious aerosols under the infection threshold.

### BRIEF DESCRIPTION OF THE DRAWINGS

FIG. 1 is a block diagram illustrating a facility immune system in accordance with an example of the present disclosure.

FIG. 2 is a floorplan of a facility employing a facility immune system in accordance with an example of the present disclosure.

FIG. 3 illustrates an area of a facility immune system in accordance with an example in which a facility immune cell includes a short-ranged beacon that interacts with mobile devices in the area.

FIG. 4 is a block diagram of an example of a facility immune cell capable of monitoring and reporting activity in the area.

FIG. 5 illustrates modules in a user interface for a facility immune system in accordance with an example of the present disclosure.

FIG. 6 is a floorplan with color coding or visual coding indicating infection statuses of facility areas.

FIGS. 7-1, 7-2, and 7-3 illustrate an example of a facility immune system using a multifunction ultraviolet disinfection system in an area to control an atmospheric load of infectious aerosols in the area.

FIG. 8-1 shows a calculate atmospheric load or risk of an infectious aerosol in a room without and with air filtering assuming a source of the infectious aerosol is present.

FIG. 8-2 shows a calculated atmospheric load or risk of an infectious aerosol in a room subject to multifunction filtering that alternates between air filtering and room-wide UV disinfection.

The drawings illustrate examples for the purpose of explanation and are not of the invention itself. Use of the same reference symbols in different figures indicates similar or identical items.

### DETAILED DESCRIPTION

People visiting, using, or working at a facility such as a hospital, a school, or other place where commercial, industrial, or service activities are conducted may leave contagions or pathogens such as bacteria or viruses on surfaces or in the air in areas of the facility. The contamination may infect the facility so that the facility can spread infections to people. In accordance with an aspect of the current disclosure, a facility immune system (FIS) provides a system for eliminating infections at facilities. In particular, an FIS may monitor contaminating and/or decontaminating activities at facilities, calculate infection statuses that identify areas where air or surface disinfection may be advisable, document cleaning and disinfection processes that remove pathogens, and enable administrative budgeting, scheduling, and monitor of disinfection processes. The FIS may further provide automated control of disinfection equipment. An FIS can thus provide an integrated solution to identify possible contagions; provide information to decision makers; budget, instruct, or schedule disinfection processes to neutralize the potential infections; and control and record disinfection activities.

An FIS may include infrastructure such as facility immune cells (FICs) that the FIS may use to monitor activities in respective areas of a facility. The FICs may be distributed throughout the facilities protected by the FIS and may operate to detect or track activity such as movement of sources of contamination such as people at the facility and detect presence and use of cleaning and disinfection resources. The FIS may further collect, analyze, and provide contamination and decontamination data for use in determining where and when disinfection processes are needed. The FIS may further include administrative and computing resources that collect and use activity data for planning efficient deployment of disinfection resources such as equipment and staff at the facility. Computing resources of an FIS may employ a main, e.g., cloud-based, computing system that provides a repository of contamination and decontamination data, a library of decontamination plans, control modules that may calendar and control decontamination processes, and computational modules that process activity data to identify the statuses of areas of facilities, e.g., areas needing decontamination, and that document areas of the facility that are safe.

Each room or area of a facility protected with an FIS may be equipped with an FIC. In one example, an FIC includes a short-range beacon, e.g., a small Bluetooth device, that repeatedly transmits location signals to near-by communication devices, which may be carried by people or decontamination equipment at the facility, and the communication devices receiving the beacon signals may periodically report to computing resources of the FIS that track or monitor activity such as the activities of people and equipment that may contaminate or decontaminate of areas of the facility. In another example, FICs may include sensors to track or monitor activity, and the FICs report activity data to the FIS computing resources that determine the statuses of the areas. An FIC may further include or be associated with a status indicator, e.g., a display or a warning light, that indicates an infection status of the area associated with the status indicator.

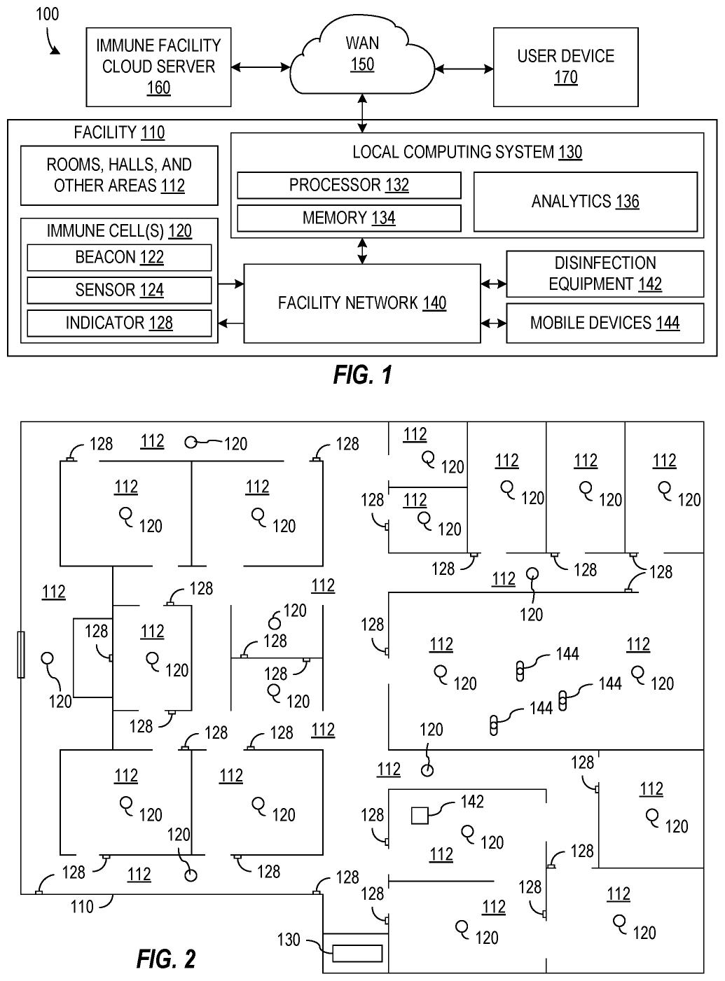

FIG. 1 is a block diagram showing some major components or features of an FIS 100 in accordance with an example of the present disclosure. FIS 100 may operate to help maintain healthy conditions in one or more facility 110. Each facility 110 includes one or more physical locations, e.g., one or more buildings, where human activities occur. A facility may, for example, be a school, a hospital, a shopping mall, an office building, a business campus, a convention center, or a hotel. Each facility 110 generally includes rooms, corridors, and other indoor or outdoor areas 112 that may contain furnishing or equipment 114 that people may visit, occupy, or use. Surfaces in areas 112 may need to be periodically serviced, cleaned, or disinfected. Similarly, the air in areas 112 of a facility may need to be filtered or disinfected to keep the atmospheric viral or bacterial loads in the areas bellow a threshold at which infections become possible or likely.

FIS 110 includes facility immune cells (FICs) 120 that are distributed in areas 112 of each facility 110. FIG. 2 shows a floorplan of one example of a facility 110 including a set of rooms, corridors, or other indoor areas 112, some of which may (or may not) be separated from each other by walls, doors, curtains, or other structures. Each area 112 in facility 110 has an associated FIC 120 that the FIS uses to monitor activity in the associated area 112. FICs 120 may be used to provide activity data to a local computing system 130. The activity data for an area 112 may indicate when people enter or leave the area 112, the durations of stays in the area 112, when equipment 142 such as disinfection equipment enters or leaves the area 112, the time at which a surface or air disinfection process was completed, or the time since completion of the last disinfection process. Local computing system 130 may collect and analyze the activity data or may report the activity data or analysis through a wide area network (WAN) 150, e.g., the Internet, to an immune facility cloud server 160 as shown in FIG. 1.

A local area network (LAN) 140, e.g., a WiFi or other wireless network, provides communication that local computing system 130 may use to receive data or communicate with devices at the facility 110. For example, depending on the implementation, local computing system 130 may communicate with one or more of FICs 120, disinfection equipment 142, and/or mobile devices 144 to determine when equipment or people enter, perform activities, or leave areas 112. Local computing system 130 may include a computer with memory 134 and a processor 132 configured to execute firmware or software 136 that implements the processes and functions disclosed herein. Local computing system 130 may particularly process activity data for local use or for transmission to remote server 160. For example, local computing system 130 may identify or track movement of people into an area 112 as possible sources of contamination of the area 112 and may accumulate the number or activity duration of possible sources of contamination in the same area 112 to identify a risk of a contagion in the area 112 or a need for disinfection of that area 112. Local computing system 130 may also document a lack of visitors to an area 112 indicating that the area 112 does not require decontamination. When people remain in an area 112, local computing system 130 may calculate or otherwise determine an atmospheric load of airborne bacteria, virus, or infections aerosols indicating when an air disinfection process may be needed. Local computing system 130 may further identify or distinguish mobile devices 144 carried by facility staff and identify the presence or use of equipment 142 in areas 112 and document the commencement and completion of cleaning or disinfection processes in areas 112. Local computing system 130 may also track times since the last decontaminations of areas 112 to determine which areas 112 may be due for decontamination or to otherwise determine infection statuses of areas 112.

In the illustrated example, facility 110 includes status lights or indicators 128 that are associated with corresponding areas 112 and may be components of FICs 128. Some or all areas 112 may have one or more associated status indicators 128 that may provide a visual indication of an infection status of the associated area 112. In one example, one or more status indicators 128 may include warning lights that local computing system 130 may control through facility network 140, e.g., to change the color of the warning lights for a particular area to indicate whether the area 112 needs disinfection or cleaning. Alternatively, each status indicator 128 may include an LED or LCD display or tablet computer which is mounted in the associated room or area 112 to indicate the infection status or need to disinfect the room. For instance, a status indicator 128 may show a green color to indicate the associated room or area 112 is safe and does not need to be disinfected, a yellow color to indicate the area presents a low risk, or a red color to indicate the associated room or area 112 needs to be disinfected.

Disinfection equipment 142 at facility 110 may employ many different technologies for surface or air disinfection. For example, equipment 142 may include germicidal ultraviolet (UVC) disinfectors, high-efficiency particulate air (HEPA) filters, needlepoint bipolar ionization (NPBI) systems, or multifunction disinfection equipment. In some examples, equipment 142 may include robotic equipment that FIS 100 is able to autonomously control to perform automated disinfection processes. U.S. patent application Ser. No. 17/246,524, entitled “Robotic Germicidal Irradiation System,” which is hereby incorporated by reference in its entirety, describes an example of a robotic UV disinfection device that may be employed in some examples of the present disclosure. Alternatively, equipment 142 may include multifunction disinfection equipment capable of operating in different disinfection modes and FIS 100 may control equipment 142 to operate (or not) in a disinfection mode that FIS 100 may select based on the infection status of the area 112 containing equipment 142.

A human administrator may control FIS 100 using a user device 170, e.g., a desktop computer, a laptop computer, a pad computer, or a smartphone, that executes a suitable application or other software providing an interface with local computing system 130 or cloud server 160. In general, user device 170 may be a mobile device 144 located at facility 110, or the administrator and user device 170 may be remote from facility 110. User device 170, for example, may be used to communicate through WAN 150 with cloud server 160 or through LAN 140 or a LoRa with local computing system 130 at a facility 110. The administrator, who may be almost anywhere, may receive notifications or other information regarding facility 110 and may provide instructions regarding decontamination procedures through user device 170.

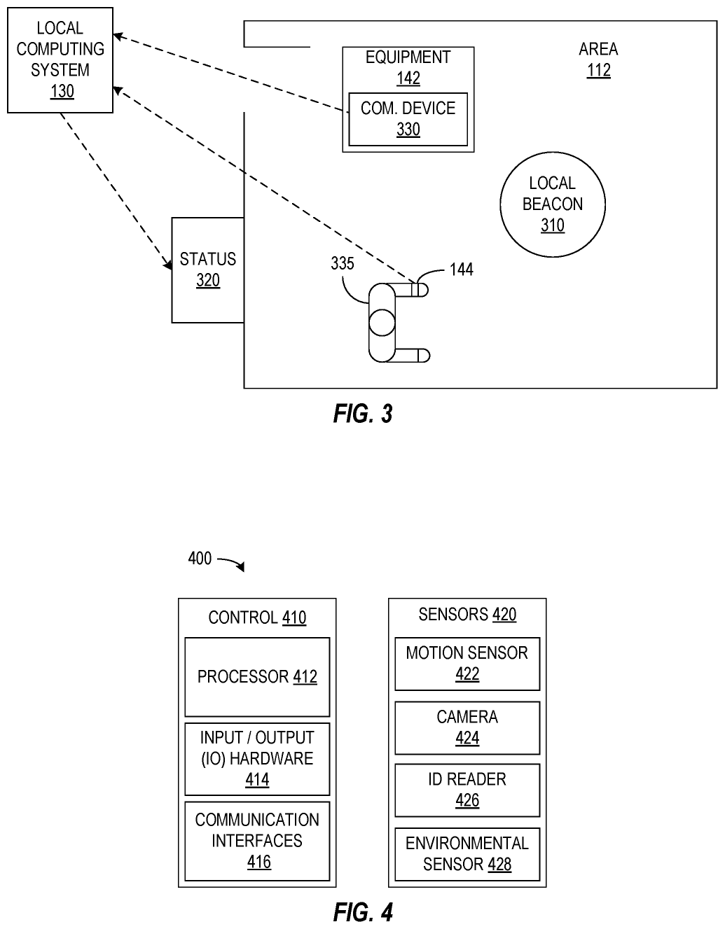

An FIS 100 may use FICs 120 to obtain activity data using different methods depending on the features and capabilities of FICs 120. In the example of FIG. 1, each immune cell 120 includes a beacon 122 and one or more sensors 124. In the example of FIG. 3, each FIC 120 includes a shortrange or local beacon 310 but may or may not have sensors. Local beacon 310 may, for example, be a Bluetooth device that transmits location information that other Bluetooth-enabled devices, e.g., equipment 142 and mobile devices 144, in area 112 can receive. In the example of FIG. 3, equipment 142, e.g., disinfection or cleaning equipment, that may temporarily be in an area 112, includes a communication device 330 that is configured to receive location information from any or the nearest beacon 310 that is within the range of a local communication protocol, e.g., Bluetooth, and communication device 330 may be configured to periodically transmit location or activity data using a network protocol, e.g., Wi-Fi, to local computing system 130. Similarly, people 335, e.g., staff, customers, students, or visitors, at the facility may carry mobile devices 144, e.g., smart phones, implementing applications that receive location information from local beacon 310 and periodically report location information or activity data to local computing system 130. As shown in FIG. 1, a tracker or other analytics module 136 implemented in local computing system 130 or elsewhere in the FIS may accumulate activity data to track where equipment 142 and people 335 were at the facility when activities, e.g., disinfection processes, were performed.

FIG. 4 shows an alternative architecture for an FIC 400. In the example of FIG. 4, FIC 400 includes a control and communication unit 410 and a set of sensors 420. Control unit 410 in one implementation is a pad computer that may be mounted in the area 112 associated with FIC 400. More generally control unit 410 may include a processor 412, input/output hardware 414, and communication interfaces 416. Processor 412 may be a general purpose processor with associated memory and software configured to implement the functions of control unit 410. IO hardware 414 may include devices such as a touch screen, keypad, microphone, speakers, or other hardware that allows staff 144 to enter or receive any type of information that may be relevant to the associated area 112. Staff 144 using IO hardware 414 may, for example, enter data identifying a type, start time, or end time of cleaning or decontamination processes performed in the area 112 associated with FIC 400. IO hardware 414 may also provide staff 144 or others with information regarding the associated area 112, e.g., an infection status, a warning, or instructions regarding cleaning or decontamination procedures. Communications interfaces 416 provide communications with other devices such as sensors 420 and local or remote computers, e.g., local computing system 130 or cloud server 160 of FIG. 1. Communication interfaces 416 may also provide a local beacon, e.g., a Bluetooth beacon as described above, that provides area information to devices in the associated area 112. In addition to processes specifically concerning the area 112 associated with FIC 400, processor 412, IO hardware 414, and communications interfaces 416 may also implement a user interface that allows an authenticated human administrator to receive information from an immune facility cloud server or to control an entire FIS.

Sensors 420 of FIC 400 may be employed to detect or measure activities and conditions in the area 112 associated with FIC 400. In the example of FIG. 4, sensors 420 include a motion sensor 422, a camera 424, an identification reader 426, and an environmental sensor 428. One function of sensors 420 is to identify activity or occupancy in the associated area 112. In particular, occupancy or motion sensing may use motion sensor 422 to detect people and equipment entering into, staying within, or leaving the area 112 associated with FIC 400. Alternatively or additionally, camera 424 may capture images or video of the associated area 112 and an AI or recognition process, e.g., in local computing system 130 or cloud server 160, may analyze the camera data to identify people or equipment entering, occupying, or exiting the associated area 112. Identification reader 426, e.g., a radio frequency identification (RFID) reader, may also determine when a person entering, working in, or leaving the associated area 112 is wearing or carrying recognizable identification or if equipment (carried by a person or otherwise) enters the area has recognizable identification, e.g., RFID tag or a visible bar code, QR code, or similar marking that reader 426 is capable of recognizing. Another function of sensors 420 may be to measure environmental conditions in the area associated with FIC 400. For example, environmental sensor 428 may measure the atmospheric temperature, humidity, light exposure, or other environmental factors in the area 112 that may affect the survival of persistence or transmissibility of contagions that may be in the area 112.

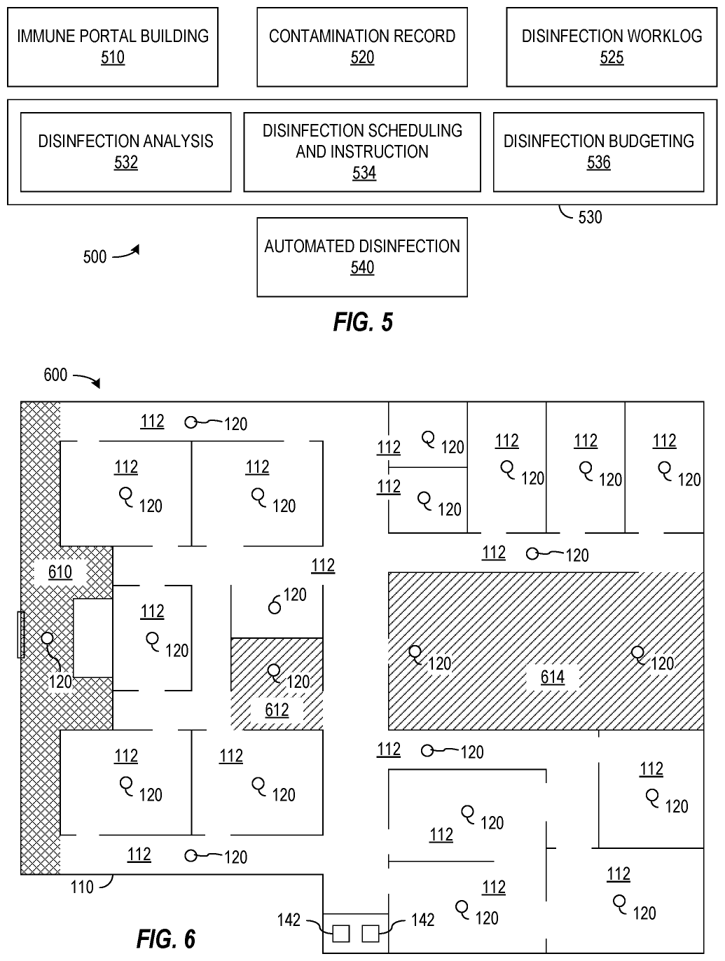

The area-specific information collected using FICs 120 of FIG. 1 may be analyzed using local computing system 130 or cloud-based server 160. Cloud-based server 160 in an example of the present disclosure provides interfaces and functions, e.g., a website, that enables an administrator to monitor, configure, and control one or more facility immune systems. FIG. 5 illustrates a user interface 500 that includes features or modules for immune portal building 510, accessing disinfection information such as a contamination record 520 or a disinfection worklog 525, disinfection planning 530, and automated disinfection performance control 540.

Immune portal building 510 provides a user interface that allows an administrator to create one or more “immune portals” respectively corresponding to one or more facilities associated with an FIS. Building an immune portal for a facility may include identifying the facility, providing a floor plan or three-dimensional model of the facility, and linking local FIS hardware at the facility with the immune portal, e.g., connecting local computing system 130 with cloud server 160 in FIS 100 of FIG. 1. Facility identification may be a process in which an administrator, e.g., a user operating user device 170 of FIG. 1, may give identifying information for a facility, e.g., a name and address or GPS coordinates of the facility, and may direct a cloud-based server to authenticate the local facility hardware. For the example of FIG. 1, an administrator using user device 170 and immune portal building module 510 of FIG. 5 may direct cloud server 160 to communicate with and commission or register local computing system 130 and identify the available FICs 120 at the facility 110 corresponding to the immune portal being built. The administrator may also provide to the cloud server 160 with a 3D model or a floorplan of facility 110 with identification of specific locations of respective FICs 120 at the facility 110. In some example FIS systems, FICs 120 may include a GPS system or may be commissioned using a GPS system that provides the coordinates of each FIC 120 to local computing system 130 or cloud server 160. Cloud server 160 may subsequently be capable of matching activity and condition data with specific areas 112 at the facility 110.

Immune portal building module 510 may further provide a user interface that allows the administrator (or the local computing system) to identify available disinfection resources at the facility and establish criterion for determining when disinfection of an area is needed. One example disinfection equipment 142 at a facility 110 may have built-in identification or intelligence that can communicate with FICs 120 for identifying when the equipment 142 is used or present in the area 112 associated with the FIC 120, so that local computing system 130 or cloud server 160 can track use of equipment 142 in respective areas 112 and record disinfection activity in disinfection worklog 525. In another example, disinfection equipment 142 may include a robot or other programmable equipment that is capable of being remotely activated or controlled to perform a disinfection process. For example, U.S. patent application Ser. No. 17/246,524, which was incorporated by reference above, discloses disinfection equipment that may be programmed to navigate areas of a facility and perform automated disinfection processes, and a control system, e.g., local computing system 130, cloud server 160, or user device 170, may be provided with codes or other information that allows the control system to activate or instruct a robotic disinfection equipment to perform a disinfection process. As described further below, automated disinfection module 540 may automatically control robotic devices. Staff at the facility 110 may also have identification that FICs 120 can read or mobile devices 144 that can communicate with FICs 120, and cloud server 160 or local computing system 130 can monitor staff identification information to determine when staff enters an area 112 to perform or initiate a disinfection process.

The administrator may further use immune portal building module 510 chose criterion for when air or surface disinfection processes should be conducted in an area of a facility or for distinguishing different possible infection statuses of an area. For example, an administrator may choose a base or maximum time between disinfection processes in each area 112, so that when the set time has passed since the last disinfection of an area 112, the area 112 may be identified as having a status indicating the area 112 presents risk and is due for disinfection. The set time between disinfections may be varied or cut short based on user-selected criterion. Such criterion may determine an infection status for each area based on factors including the number or duration of unidentified (or identified) visits to an area 112 since the area was last disinfected. For example, a disinfection analysis module 532 may set the infection status of an area 112 to clean (code green) and reset an entry for the area 112 in contamination record 520 when a disinfection process is determined or reported to be completed. After that, the area's entry in contamination record may indicate a count of people detected entering an area 112 or durations of occupancy of the area 112. If the total time that people spend in an area 112 reaches a threshold level, analysis module 532 may update the infection status of the area 112 to indicate the area 112 has a low risk of being contamination (code yellow) or to indicate the area is due for disinfection (code red). In one example, the cloud server 160 may separately tabulate in contamination record 520 the numbers and the durations of stays of people that enter each area 112 of FICs 120, and areas 112 having the higher traffic or longer occupation may be identified as having higher priorities for disinfection processes. As noted above, since FICs 120 may also be used to track or monitor disinfection processes, an FIS may reset contamination data for an area after completion of a disinfection process in the area.

Disinfection planning module 530 may provide several functional modules that an administrator (human or AI) may use to plan disinfection processes. In the example of FIG. 5, disinfection planning module 530 includes a disinfection analysis module 532, a disinfection scheduling and instruction module 534, and a disinfection budgeting module 536. Disinfection analysis module 532 may analyze activity and condition data and parameters relevant to areas of a facility determine infection statuses for the areas. Based on the infection statuses, a disinfection scheduling and instruction module 534 may be used to set up or schedule cleaning or disinfection processes in areas of the facility having a higher risk status and to provide instructions regarding how the disinfection should be completed. Module 534 may automatically schedule disinfection, e.g., send instructions to staff or automated disinfection module 540, or may contact an administrator that approves or schedules disinfection processes. Disinfection scheduling may include instructions on how staff or automated equipment should perform the disinfection process. For example, having a 3D model or floorplan of a facility, the cloud server 500 implementing module 534 can calculate positioning and exposure times for use of UV disinfection equipment during a disinfection process for an area. U.S. patent application Ser. No. 17/092,010, entitled “Ultraviolet Germicidal Irradiation Room Analysis,” which is hereby incorporated by reference in its entirety, describes some processes for using 3D models to determine efficient and effective plans for surface disinfection of an area, and cloud server 160 may use such process to provide an administrator, staff, or disinfection equipment with instructions for efficiently disinfecting areas in the facility. Disinfection planning feature 530 may further permit an administrator (or AI of cloud server 160) to use disinfection scheduling module 534 to schedule times and locations for disinfection process and may communicate the schedules to facility staff or intelligent equipment. A disinfection budgeting module 536 may provide an administrator with cost estimates or budgets for use of cleaning or disinfection equipment in a disinfection process.

An FIS, e.g., analysis module 532, may determine the infection statuses of areas 112 using current measured area data, e.g., area activity data and condition data, and relatively fixed parameters. Some examples of relevant parameters may be distinguished as being engineering parameters, physical parameters, and epidemiological parameters.

Engineering parameters that an FIS uses generally relate to the characteristics of areas at a facility. An administrator may set or give engineering parameters for area to the FIS during immune portal building. For example, some typical engineering parameters of an area 112 may include fixed parameters such as a room volume, floor surface area, and a mean ceiling height. Engineering parameters may further include operating parameters of systems or equipment in areas 112. These engineering parameters may, for example, relate to heating, ventilation, and air conditioning (HVAC) or air filtration systems and include an outflow rate from the area; a ventilation (outdoor air exchange) rate; a recirculation or interior air exchange rate; a probability of droplet or particulate filtration via recirculation; a filtration removal rate; and UVC disinfection rate of systems in the areas. Engineering parameters may also reflect devices or protection that people may employ in the area. For example, if people are required to wear masks in an area, a mask penetration probability may be an engineering parameter relevant to determining the infection status or risk of the area.

Physical parameters that an FIS uses generally relate to the characteristics of one or more target pathogens or sources of the target pathogens. For example, physical parameters relevant to a pathogen that can be distributed through respiration may include parameters indicating the characteristics of respiration that may carry the pathogen, e.g., a respiratory drop radius 0.1-100 m; a drop volume; a drop number density per radius; EB drop settling speed; drop settling rate; mean breathing flow rate; background CO₂ concentration; exhaled CO₂ concentration; and production rate of exhaled CO₂.

Epidemiological parameters that an FIS use may relate to current characteristics of the general population or to carriers of a currently prevalent or targeted pathogen. Examples of epidemiological parameters may include: numbers or percentage of susceptible and infected persons that may be present at the facility or area; an airborne transmission rate per infected-susceptible pair; pathogen (virion) deactivation rate; pathogen concentration relaxation rate; effective infectious drop radius; pathogen production rate/air volume/drop radius; infectious pathogen concentration/air volume/radius; pathogen (virion) concentration per drop volume; pathogen infectivity; infectiousness of breath, exhaled quanta concentration; quanta emission rate; probability a person is infected (prevalence); probability a person is immune (by vaccination or exposure); probability a person is susceptible; BA relative susceptibility (or transmissibility); expected number of infected-susceptible pairs; expected number of airborne transmissions; and indoor reproductive number.

Epidemiological parameters generally change depending on which pathogens are currently prevalent and the status of the population generally or at the facility. In accordance with an aspect of the present disclosure, an FIS, e.g., disinfection analysis module 532, may periodically update epidemiological parameters from authoritative sources, e.g., using information provided by the Center for Disease Control (CDC) so that the infection status of areas are automatically determined based on best available information. Accordingly, an FIS may relieve an administrator of the need to constantly look up and change contamination procedures as best information and practices for safe disinfection change.

Disinfection worklog 525 may be based on reports or sensing of completion of disinfection processes and further provides a record documenting the disinfection processes. An administrator may use worklog 525 to review disinfections that have been performed and identify disinfection process that have been completed, identify historical patterns or trends in the disinfections processes, and anticipate future disinfection process that may be needed at a facility. Disinfection worklog functionality 525 of user interface 500 may, for example, present an administrator with a floorplan on a smartphone display with color coding as shown in FIG. 6 to identify areas 610, 612, and 614 that may be in greatest need of disinfection. FIG. 6 illustrates an example of a floor plan in which areas 610, 612, and 614 in facility 110 may statuses indicating a risk or need of disinfection, and color coding of the areas 610, 612, and 614 may indicate calculated levels of exposure that areas 610, 612, and 614 may have had since last being disinfected. The status or the need to be disinfected or levels of exposure in each room, may be displayed on the digital floor plan of the user interface 130, or be displayed on an LED indicator or LCD display which is posted in every room and connected to the immune facility cloud server 160, e.g., using status indicators 128 shown in FIGS. 1 and 2.

An FIS may be used to control surface and airborne pathogens and to actively monitor the safety of a facility. FIGS. 7-1, 7-2, and 7-3 illustrate how an FIS 700 may be used to prevent or reduce transmission of airborne pathogens at a facility including a room 710. Room 710 is an area that has an associated FIC 720 that a computing system 710, e.g., a local computing system 130 or a cloud server uses to monitor contamination and disinfection processes in room 710. A shown, room 710 is equipped with a multifunction UV disinfector 740, which may be portable equipment currently in room 710 or may be a fixture permanently installed in room 740. Multifunction UV disinfector 740 has multiple operating modes. One mode is an air filtration mode, which may be safely used when room 710 is occupied, e.g., while people 750 are in room 710, and another mode is a UV irradiation mode, which may be most safely conducted when room 710 is vacant. U.S. patent application Ser. No. 17/138,332, entitled “Multifunction UV Disinfector” or U.S. patent application Ser. No. 17/161,404, entitled “Multifunction UV Disinfecting Fixture,” which are hereby incorporated by reference in their entirety, further describe examples of multifunction UV disinfectors.

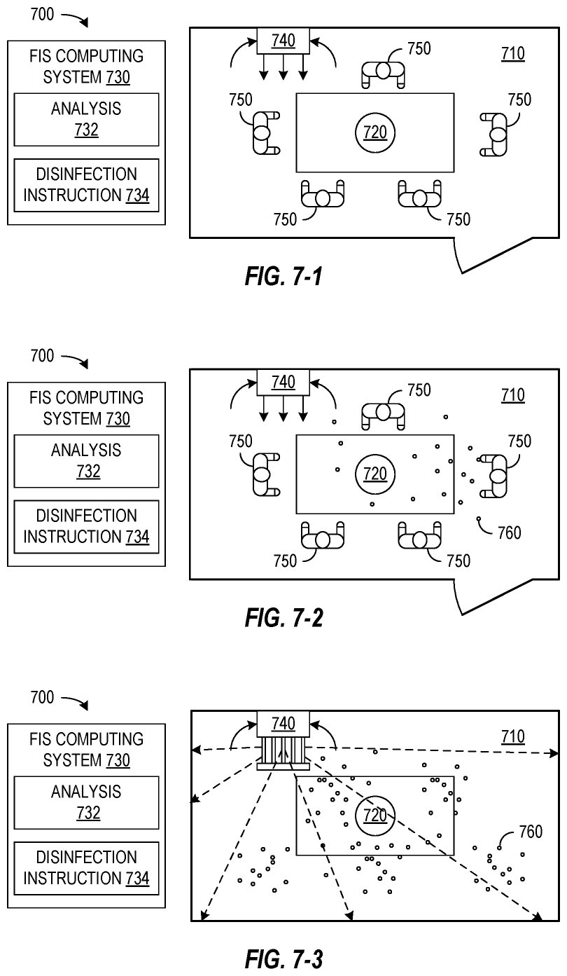

FIG. 7-1 illustrates a situation in which room 710 has a safe infection status, which may occur immediately after a disinfection process was completed in room 710. Surfaces and the air in room 710 have at most low levels of pathogens that provide no or a low probability of transmission of the pathogens. Room 710 is safe for normal activities as shown in FIG. 7-1, and people 750 may occupy room and undertake normal activities, e.g., teaching and learning in a classroom or discussion at a meeting in a conference room. During this time, FIS computing system 730 acquires activity data indicating the occupation of room 710. For example, computing system 730 through use of FIC 710 as described above may collect data indicating the number of people occupying room 710 and the duration of stays of each person or of people 750 collectively. Computing system 730 may further be able to identify people 750 or results of tests of people 750 for a target pathogen or contagion.

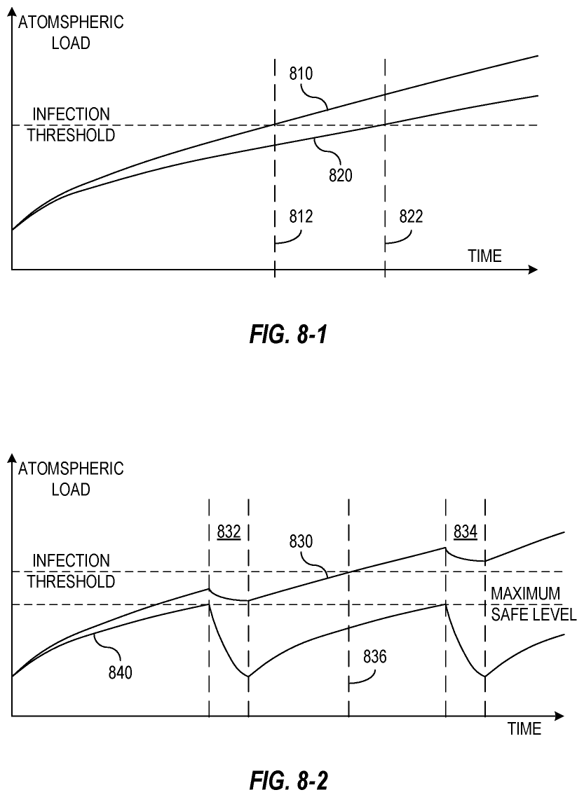

FIG. 7-2 shows an example where one or more person 750 in room 710 is a source of a contagion and produces infectious aerosols 760. Initially, even with a contagious person in room 710, the concentration or load of airborne contagion 760 remains low, and the low load of airborne contagions is relatively safe for the people 750 who are susceptible to but not infected by the contagion. Transmission of many diseases caused by airborne contagions may be unlikely while the load of airborne contagions is below an infection threshold. FIG. 8-1 shows the time dependence of the concentration or load 810 of a contagion in an area, e.g., room 710, without active air filtration or disinfection. Load 810 in room 710 increases over time if a contagious person remains in room 710, and at time 812, load 810 rises above an infection threshold at which infection of susceptible individuals becomes likely. An analysis module 732 implemented in computing system 730 may calculate or estimate the load, e.g., load 810, in room 710 based on assumptions that one or more people 750 may be contagious, and disinfection instruction module 734 may take action to warn people 750 or operate disinfection equipment 740 in room 710 based on the room analysis.

Disinfection equipment 740 may be operated in air filtration mode to remove contagions from the air in room 710. Disinfection equipment 740 operating in air filtration mode generally draws air from room 710, removes or deactivates a fraction of the contagions, e.g., infectious aerosols, from the drawn air, and returns filtered or clean air back to room 710. Air filtration may slow or stop the rate of increase air borne contagions in room 710. In general, the atmospheric load may at least initially increase when a contagious person enters the room even if air filtering is active, but the rate of increase in the atmospheric load in the room will generally be less when air filtering is active. Depending on the efficiency of the air filtration or disinfection, the atmospheric load of the infectious aerosols may or may not reach the infection threshold. FIG. 8-1 shows time dependence of an atmospheric load 810 or 820 in a room if air filtration is not used or is used while a contagious person is in the room. In the illustrated example, load 820 rises more slowly than load 810, so that the room remains safe for a longer time, e.g., until a time 822, which is after the time 812 when the infection threshold is reach without filtering. But, at time 822, load 820 rises above the infection threshold making the room unsafe. In the system of FIG. 7-2, an analysis module 732 of FIS computing system 730 can calculate the infection status of room 710 taking into account engineering, physical, and epidemiological parameters relevant to room 710 and the target contagion. If the calculated infection status of room 710 rise to a level where a decontamination process is need, e.g., near the infection threshold, FIS 730 may warn people 750 to leave room 710 before the infection threshold is reached.

A multifunction disinfector in an area can switch to a more effective or “reset” disinfection mode, e.g., UV disinfection process, when people have left the area. FIG. 7-3 shows an example where people 750 have left room 710, and multifunction disinfector 740 conducts a reset disinfection process. In particular, a disinfection instruction module 734 of computing system 730 can instruct multifunction disinfector 740 to activate a UV irradiation mode. In the UV irradiation mode, UV lamps that are shielded with disinfector 740 when disinfector 740 acts only as an air filter, may move out from the shield to irradiate the air (and surfaces) throughout room 710 to quickly disinfect the air and surfaces room 710. At the same time, air circulation may continue to move air in room 710 toward UV disinfector 740. UV disinfector 740 may continue the “reset” disinfection process for a time that FIS computing system 730 determined is needed to return room 710 to a safe status.

FIG. 8-2 shows an example plot of time dependence of an atmospheric load 840 in a room in which an FIS operates a multifunction disinfector. The multifunction disinfector, e.g., disinfector 740 in FIG. 7-2, may initially operate in an air filtration mode, during which load 840 increases in the same manner as in load 820 described above. During a time interval 832, which is before load 840 reaches the infection threshold, people leave the room, and the multifunction disinfector, e.g., multifunction disinfector 740 in FIG. 7-3, operates in a UV disinfection mode, e.g., irradiates the air and surfaces of the room with UVC radiation. The UV disinfection quickly reduces (or resets) atmospheric load 840 to a safe level before the multifunction disinfector returns to air-filtration-only mode and people return to the room. Contagion load 840 may then increase (if a contagious person is present) until a time interval 834 when people again leave the room and the multifunction disinfector switches again begins UV disinfection of the room. The cycle of switching between only air filtering and UV disinfection (with or without simultaneous air filtering) can be repeated indefinitely so that load 830 never rises above the infection threshold and people using the room remain safe.

Some facilities have regular periods in which an area, e.g., room 710 of FIG. 7-1, 7-2, or 7-3, may be unoccupied. For example, if room 710 is a classroom at a school, then classroom 710 may be unoccupied during lunch or recess periods. In accordance with another aspect of the present disclosure, FIS 700 schedule a multifunction UV disinfector 740 to operate in UV irradiation mode during periods when room 710 is normally unoccupied, and during the scheduled times, multifunction UV disinfector 740 can operate in UV irradiation mode if FIS 700 and disinfector 740 measures and confirms that room 710 is unoccupied.

When the buildup of infectious aerosol concentration is faster than the total air exchange rates of HVAC and air purification, a quick “reset” of the concentration of infectious aerosols in the room is periodically needed, so that infectious aerosol concentration never exceeds the threshold concentration throughout the whole gathering process. As shown in FIG. 8-1, when the buildup of infectious aerosols is faster than the total air exchanging speed of HVAC and air purification, the infectious aerosol concentration 810 or 820 increases with time and eventually and exceeds the threshold value of infection at time 812 or 822 depending on the operation of HVAC or filter systems for the room. However, in FIG. 8-2, if a “reset” processes using UV irradiation of the entire room is performed during a time interval 832, e.g., during a break time, the effective build-up time of the aerosol concentration is shortened, and the risk of the concentration exceeding the threshold of infection is reduced.

Another example occurs when a classroom is open during normal school hours, e.g., 8 AM to 5 PM. If the classroom is continuously occupied during the entire time, the maximum infectious aerosol concentration may become very high and could exceed the infection threshold concentration at as illustrated in FIG. 8-1. A calculated infectious aerosol concentration 830 illustrates a case in which the classroom is unoccupied during time intervals 832 and 834, e.g., during recesses or a lunch period. As shown, during intervals 832 and 834, no people (or sources of aerosols) are present, and infectious aerosol concentration 830 decreases, e.g., as a result of HVAC operation, filter operation, or natural inactivation of the aerosols. However, when people return, infectious aerosol concentration 830 may still rise above the infection threshold at a time 836. In contrast, a lower infectious aerosol concentration 840 may be maintained if “reset” processes, e.g., room-wide UV irradiation processes, are performed during breaks 832 and 834. The minimum frequency of executing such “reset” processes depends upon the speed of infectious aerosol build-up. For example, a “reset” process may only be executed during the lunch break, or every two hours, or every hour, depending on the needs and convenience.

One example of a process to “reset” infectious aerosol concentration uses a “ventilated UVC aerosol disinfection system.” The disinfection system includes a UVC light source, a ventilation system to pull room air near the UVC light source. Conventional UVC disinfection systems are designed to disinfect the surfaces in a room. UVC surface disinfection may be relatively time consuming because (1) the beam angel of the UVC light beam with respect to the surfaces may not be optimal for all surfaces in the room, (2) the distance from the UVC light source to some surfaces may be far, and (3) obstructions shadow or block the UVC light from some surfaces. However, for an aerosol reset process, UVC is used to disinfect aerosols. All the UVC light may be beamed to any aerosols with direct angle. Also, all the aerosols at far distance can be drawn or pulled near the UVC light source using a fan or other air moving system to increase the UVC dosage reaching the entire air volume and shorten the disinfection time. Similarly, aerosols hidden behind the shadows, can be pulled out of the shadows, and toward the UVC light sources by the integrated ventilation system. (Although ventilation can speed up the aerosol disinfection, a ventilation system in the UVC system is optional. For example, even if there is no integrated ventilation system, UVC aerosol disinfection may still work due to the ventilation provided by the existing HVAC and/or air purification systems. The ventilated UVC aerosol disinfection or reset process may only be executed when the room is un-occupied because UVC light might be harmful to human eyes. Since most gatherings have some kinds of break times, such as lunch breaks, recess times, reset processes may be easy to find a 5 to 10 minute break time to allow the “reset” process executed without significant interruptions of gatherings.

Each of modules disclosed herein may include, for example, hardware devices including electronic circuitry for implementing the functionality described herein. In addition or as an alternative, each module may be partly or fully implemented by a processor executing instructions encoded on a machine-readable storage medium.

All or portions of some of the above-described systems and methods can be implemented in a computer-readable media, e.g., a non-transient media, such as an optical or magnetic disk, a memory card, or other solid state storage containing instructions that a computing device can execute to perform specific processes that are described herein. Such media may further be or be contained in a server or other device connected to a network such as the Internet that provides for the downloading of data and executable instructions.

Although particular implementations have been disclosed, these implementations are only examples and should not be taken as limitations. Various adaptations and combinations of features of the implementations disclosed are within the scope of the following claims.

What is claimed is:

1. A system comprising:
a computer system configured to determine a plurality of statuses respectively for a plurality of areas at a facility, the statuses indicating respective risks of a contagion in the areas; and
a plurality of cells respectively in the areas at the facility, the cells operating to provide the computer system with data indicating activity in the areas, the computer system using the data to determine the statuses.

2. The system of claim 1, wherein each of the cells comprises a sensor system that senses the area of the cell and produce sensor data indicating conditions in the area containing the cell, the computer system further using the sensor data to determine the statuses.

3. The system of claim 1, wherein each of the cells comprises a sensor system that senses the activity in the area containing the cell.

4. The system of claim 3, wherein each of the sensor systems comprises an occupancy sensor configured to detect when the area containing the cell is occupied, the computer system determining the statuses using data that the occupancy sensors provide.

5. The system of claim 1, wherein each of the cells comprises an identification reader capable of reading identification on staff or equipment when the staff or equipment is in the area containing the cell, the computer system determining the statuses using data that the identification readers provide.

6. The system of claim 1, wherein the activity data indicates when people enter and leave the areas, and the computer system calculates the risks based on times people spend in the areas.

7. The system of claim 1, wherein the activity data indicates when disinfection processes are completed in the areas of the facility.

8. The system of claim 7, wherein the computer system calculates the risks based on times since the disinfection processes were last completed in the areas

9. The system of claim 1, wherein each of the cells a beacon that transmits to mobile devices in the area of the cell, the mobile devices transmitting the activity data to the computer system in response to communication from the beacon.

10. The system of claim 1, further comprising a worklog maintained by the computer system and indicating when and where the disinfection processes were completed.

11. The system of claim 1, wherein the computer system is further configured to generate instructions for one or more disinfection processes to be performed in one or more of the areas of the facilities.

12. The system of claim 11, wherein the computer system generates the instructions in response to the risks in the one or more areas reaching a threshold risk level.

13. The system of claim 11, wherein the computer system when generating the instructions uses a three-dimensional model of the one or more areas to determine exposure times required for a UV disinfection device to disinfect surfaces in the one or more areas.

14. The system of claim 11, wherein the computer system controls disinfection equipment in the one or more areas to implement the disinfection processes.

15. The system of claim 1, wherein the computer system comprises:
a local computing system connected to the cells through a local network; and
a cloud server system connected to the local computing system, the cloud server providing an interface for an administrator to monitor and schedule cleaning or disinfection processes in the areas of the facility.

16. The system of claim 1, wherein the computer system is configured to:
operate a disinfection unit to begin a disinfection process in a first of the areas in response to identifying that the status of the first areas indicates the risk in the first area is above a threshold level; and
end the disinfection process when the disinfection processes is completed.

17. The system of claim 1, where in the computer system is configured to send a notification in response to the status of any of the areas indicating the risk is above a threshold level.

18. The system of claim 17, the notification is sent through text messaging, emails, smartphone apps, or displays that notify people of the status.

19. A system comprising:
a computer system configured to determine a plurality of statuses respectively for a plurality of areas at a facility, the statuses indicating respective risks of a contagion in the areas; and
a plurality of cells that are respectively in the areas at the facility and are connected to communicate with the computer system, each of the cells including a status indicator configured for the computer system to operate to provide notice of the status for the area containing the cell.

20. The system of claim 19, wherein the computer system maintains records of times when each of the areas were last disinfected and uses the record of the times in determining the statuses.

21. The system of claim 19, wherein the computer system is further configured to generate instructions for one or more disinfection processes to be performed in one or more of the areas of the facilities.

22. The system of claim 21, wherein the computer system generates the instructions in response to the statuses of the one or more areas reaching a threshold risk level.

23. The system of claim 21, wherein the computer system controls disinfection equipment in the one or more areas to implement the disinfection processes.

24. A process comprising:
operating an air system in a room to remove a portion of infectious aerosols from the room while people occupy the room; and
performing a UV disinfection in the room to deactivate the infectious aerosols when people leave the room.

25. The process of claim 24, further comprising:
operating a computer system to calculate a time when a risk from infectious aerosols in the room will reach a threshold level assuming that the air system continues to operate in the room and one or more of the people are contagious; and
the computer system warning the people to leave the room in response to people remaining in the room until the calculated time;

26. The process of claim 24, wherein operating the air system comprises operating a multifunction disinfector in an air filtration mode, and performing the UV disinfection comprises operating the multifunction disinfector in a UV disinfection mode.

27. The process of claim 24, further comprising people returning to the room after the UV disinfection is complete.

28. The process of claim 24, wherein:
the room comprises a classroom;
operating the air system occurs during instruction; and
performing the UV disinfection occurs during a recess or a lunch period in which the classroom is unoccupied.

29. The process of claim 24, wherein operating the air system comprises operating one of an HVAC system and a filter system.

30. The process of claim 24, wherein performing the UV disinfection comprises directing radiation from a UV source into the air and toward surfaces in the room while moving the air in the room toward the UV source.
